(19) United States
(12) Patent Application Publication
(10) Pub. No.: US 2025/0271344 A1
(43) Pub. Date: Aug. 28, 2025

(54) METHOD FOR ANALYZING THE COMPOSITION OF CIGARETTE LEAF GROUPS

(71) Applicant: CHINA TOBACCO YUNNAN INDUSTRIAL CO., LTD., Kunming (CN)

(72) Inventors: Ji YANG, Kunming (CN); Jianjun XIA, Kunming (CN); Baokun ZHU, Kunming (CN); Wei ZHANG, Kunming (CN); Qianxu YANG, Kunming (CN); Qian GAO, Kunming (CN); Shiyun TANG, Kunming (CN); Wei JIANG, Kunming (CN); Juan LI, Kunming (CN); Chunbo LIU, Kunming (CN); Chunxia YU, Kunming (CN); Kai WU, Kunming (CN); Zhenjie LI, Kunming (CN)

(21) Appl. No.: 19/007,592

(22) Filed: Jan. 2, 2025

Related U.S. Application Data
(63) Continuation of application No. PCT/CN2024/078294, filed on Feb. 23, 2024.

(51) Int. Cl. G01N 5/04; G01N 33/00

(52) U.S. Cl. CPC G01N 5/04; G01N 33/0098

(57) ABSTRACT

The invention concerns a method for analyzing a cigarette leaf composition, including the following steps: (1) preparation of a cigarette sample to be analyzed and single-grade tobacco leaf samples; and (2) collection of a thermal analysis spectrum to obtain the composition and proportion of the tobacco leaves in the cigarette to be analyzed. The method can complete the analysis of the composition of finished cigarettes in a few minutes, and can obtain a clear formula of the composition and proportion values. It is objective, efficient, highly sensitive, versatile, and has good repeatability. It has unique advantages in the analysis of finished cigarette compositions in the tobacco industry.

### CROSS-REFERENCE TO RELATED APPLICATIONS

This application is a continuation of International Pat. Appl. No. PCT/CN2024/078294, filed on Feb. 23, 2024, incorporated herein by reference as if fully set forth herein.

### TECHNICAL FIELD

The invention belongs to the technical field of tobacco, in particular to a method for analyzing the composition of cigarette leaf groups.

### BACKGROUND

The quality and style characteristics of cigarettes are mainly formed by product designers through the proportions of tobacco leaves of different origins, varieties and grades. It is usually necessary to rely on formula experience and sensory evaluation, and manually select 10-20 kinds of tobacco leaves from hundreds of stock grades of tobacco raw materials to design different formulas in different proportions. As a result, the composition of the coiled-tobacco leaf groups is extremely complicated, and it is difficult to analyze the composition of the unknown coiled-tobacco leaf groups manually. It is of great significance for the analysis of unknown tobacco and the design of leaf groups to analyze the composition of coiled tobacco leaves by means of instrument testing and using objective data and scientific technology.

Thermogravimetric analysis (TG/DTA) can provide stable reaction conditions under programmed temperature conditions, and is the most ideal experimental tool for tobacco pyrolysis research. Derivative thermogravimetric methodology, also called the derivative thermogravimetric method, is derived from thermogravimetric analysis, and is a technique to record the first derivative of a TG curve with respect to temperature or time. The result of the experiment is a derivative thermogravimetric curve, that is, a DTG curve. The characteristics of DTG curves are: accurate reflection of the initial reaction temperature, maximum reaction rate temperature and reaction termination temperature of each weight loss stage; and the area of each peak on the DTG curve is proportional to the corresponding sample weight loss on the TG curve. When the TG curve is not obvious to some steps in the heating process, the DTG curve can be clearly distinguished. The main feature of thermogravimetric analysis is that it is highly quantitative and can accurately measure the mass change and the rate of change. It can be said that according to this feature, as long as the mass of a substance changes when it is heated, it can be studied by thermogravimetric analysis.

At present, the composition analysis of coiled tobacco leaves mostly adopts the combination of tobacco chemical composition analysis, flue gas chemical composition analysis, sensory evaluation and other means. The work intensity is high, the subjectivity is strong, and the conclusions drawn are vague and not referential.

In order to solve the above problems, the invention is proposed.

### SUMMARY

Since DTG curves can effectively represent the quality information of cut tobacco/tobacco leaves, the consistency between the analytical composition of the formula and the real leaf formula can be simulated and evaluated by measuring the difference(s) in DTG curves. In order to improve the generality of cigarette tobacco leaf group analysis and the workload of analysts, the invention uses a DTG differential correlation model and a combination optimization algorithm of formula analysis to analyze and characterize the quality information of cigarette cut tobacco by using a thermal analysis atlas, which can automatically search the ratio of tobacco leaf groups in a formula, and which analyzes the composition of cigarette tobacco using objective data. The composition and proportion of leaf groups in a formula can be clearly obtained, which is of great significance to the analysis and design of leaf group formulas of competing cigarettes.

The invention provides a method for analyzing a tobacco leaf group composition (e.g., in a formula for cigarettes). The method comprises analyzing the specific composition and formula ratio of tobacco (e.g., competing cigarettes to be analyzed) based on the ability to characterize tobacco quality.

The technical scheme of the invention is as follows:

A method for analyzing the composition and proportion of tobacco leaf groups in a cigarette sample based on thermal analysis includes the following steps: (1) preparing a cigarette sample to be analyzed and one or more single-grade tobacco leaf samples; (2) constructing a thermal analysis spectrum for the cigarette sample to be analyzed and the single-grade tobacco leaf sample(s); and (3) analyzing the thermal analysis spectrum to obtain the composition and proportion of tobacco leaves in the cigarette sample to be analyzed.

Preferably, in Step (1), there is a single cigarette sample to be analyzed, and at least 50 single-grade tobacco leaf samples prepared and/or analyzed. Each sample may be placed in a constant temperature and humidity environment of (22±1)° C. and (60±2) % relative humidity for at least 48 hours for equilibrium. Generally, no less than 50 typical single-grade tobacco leaf samples are selected, and the single-grade tobacco leaf samples should be of different grades, different origins and different parts (e.g., of the tobacco plant), and the smoking taste of typical single-grade tobacco samples is quite different. The tobacco sample should not be less than 5 g, and the sample crushing mesh (e.g., screen) should not be less than 100 mesh.

Preferably, Step (2), collecting the thermal analysis spectrum, includes the following sub-steps: the samples are respectively heated in thermogravimetric (TG) crucibles by a procedure including: an initial temperature of 50° C., heating at a rate of 10° C./min to a final temperature of 900° C., and maintaining temperature at 900° C. for 5 min, using a protection gas and reaction gas comprising nitrogen, at a flow rate of 20 mL/min. Taking temperature (° C.) as the X-axis and mass change (%) as the Y-axis, the exported data (e.g., a graph of the change in mass of the sample plotted as a function of temperature) is TG result data. Before sample analysis, the thermogravimetric analyzer may be set and kept at 900° C. for 10 min to clear the impurities in the selected thermogravimetric (e.g., alumina) crucible, and the empty crucible may be used as a reference. The instrument balance sensitivity of the thermogravimetric analyzer should be not less than 0.1 μg, and the curve resolution should be not less than 50 million resolution points.

Preferably, Step (3), analyzing the thermal analysis spectrum and obtaining the composition and proportion of tobacco in the cigarette to be analyzed, comprises the sub-steps:
Sub-step (A): calculating the first derivative of the TG result data (e.g., with respect to time) to obtain a differential weight loss DTG curve, a DTG matrix Y of the cigarette sample to be analyzed, and a DTG matrix of the single-grade tobacco leaves X=[X₁ X₂ . . . Xₙ], wherein n is the number of single-grade tobacco leaf samples;
Sub-step (B): coding formula proportions: coding a real number R=[r₁ r₂ . . . rₙ] for a proportion of each single grade tobacco leaf in one or more formulas (e.g., in each of a plurality of formulas), wherein n is the number of single-grade tobacco leaf samples;
Sub-step (C): randomly initializing a coding matrix R: initializing an r value to a real value between 0 and 1, wherein a sum of the values of each coding matrix should be 1, establishing a search space (e.g., a library) according to a range of (e.g., including) more than 10 times the number of tobacco leaves composed of (e.g., in) the formula (e.g., a typical cigarette tobacco formula), and randomly initializing the coding matrix (that is, for example, R₁, R₂, . . . ). Since the number of tobacco leaves in a typical cigarette formula is generally 10-20, the search space according to the 10-fold (minimum) range includes at least 200 coding matrices (e.g., R₁, R₂, . . . R₂₀₀);
Sub-step (D): calculating a DTG matrix Z after generating or calculating one or more combinations of single-grade tobacco leaves according to formula ratio R (e.g., according to the formula ratio [s] R₁, R₂, . . . R₂₀₀ in the coding matrix R), thereby obtaining at least 200 possible formula ratio candidates;
Sub-step (E): calculating a difference value e between Z and Y using a DTG differential correlation model;
Sub-step (F): converting the difference value e to a probability value P(e);
Sub-step (G): according to the probability value, screening a number of formula ratios or proportions (e.g., from among R₁, R₂, . . . R₂₀₀) to participate in a next iteration, randomly selecting two schemes (e.g., proportions of single grade tobacco leaves) for linear reorganization (or regression): r⁽¹⁾=r₁+a*(r₁-r₂), and obtaining reorganized (or regressed) real number coding matrices R₁⁽¹⁾, R₂⁽¹⁾, . . . , wherein a is a scale factor generated by random numbers that obey a [−d, 1+d] uniform distribution, and d is a value that limits the scope of the reorganization (or regression);
Sub-step (H): repeating sub-steps (C)-(F) (e.g., iterative searching), and iteratively calculating the difference value e (e.g., to generate e⁽²⁾, e⁽³⁾, e⁽⁴⁾, e⁽⁵⁾ . . . on successive calculations) until e is less than a certain value; and
Sub-step (I): ordering the probability value P(e) from largest to smallest, and taking a number of formula ratios or proportions (e.g., ranking a subset of the formula ratios R₁, R₂, . . . according to the corresponding probability value P(e) from largest to smallest), to obtain the tobacco composition and proportion of the tobacco (e.g., cigarette sample) to be analyzed (e.g., the formula ratio having the largest corresponding probability value P(e)).

Preferably, sub-step (C) should ensure that the sum of the values of each encoding matrix is 1, using an initialization formula as follows:

$${r}_{i} = {r}_{i} / {\sum }_{i = 1}^{n} {r}_{i} .$$

Preferably, in sub-step (D), to calculate the cigarette DTG matrix Z after combining single-grade tobacco leaves according to the formula ratio R, the calculation formula is as follows: Zᵢ=X′×Rᵢ, where Rᵢ is the i-th random coding matrix, X (e.g., X′) is the DTG matrix of the single-grade tobacco leaves, and Zᵢ is the DTG matrix of the formula ratio or proportion Rᵢ.

Preferably, the formula for calculating the difference value e in sub-step (E) is as follows: e=\sqrt{(Z−Y)Σ⁻¹(Z−Y)}, where Y is the DTG matrix of the cigarette sample to be analyzed, Z is the DTG matrix of tobacco leaves in proportion (e.g., according to the formula ratio Ri), and Σ is a covariance matrix between Y and Z.

Preferably, in sub-step (F), the calculation formula to convert the difference value e to the probability value P(e) between 0 and 1 is:

$$P ( e ) = \frac{{e}_{max} - e}{{e}_{max} - {e}_{min}} / sum ( \frac{{e}_{max} - e}{{e}_{max} - {e}_{min}} ) .$$

Preferably, d has a value of 0.2-0.3 in sub-step (G). To limit the scope of reorganization or regression, the value of d is generally 0.25.

Preferably, in sub-step (H), e is iteratively calculated until it is <0.0001.

The invention has the following beneficial effects:

1. The method of the present invention uses a DTG differential correlation model and a formula analysis combination and/or optimization algorithm, and automatically searches for the tobacco leaf ratio in the tobacco leaf group formulas (e.g., the library of formula ratios in the coding matrix). The method can complete the composition analysis of any tobacco sample (e.g., finished cigarette in the market to be analyzed) within a few minutes, and obtain a clear formula composition and proportion value. The method is objective, efficient, versatile, and has good repeatability and high sensitivity. It has a unique advantage in the analysis of finished cigarettes in the tobacco industry.

2. The method of the present invention avoids wet chemical analysis of a large amount of tobacco, such as in conventional flue gas chemical composition analysis of tobacco leaf groups, and turns to dry chemical operation, which has the advantages of simplicity, minimal sample usage, accuracy (e.g., within 10 mg), is non-toxic and harmless, and causes no harm to the operator and no environmental pollution.

3. The method of the present invention not only provides a thermal analysis spectrum of finished cigarettes to be analyzed (e.g., an analysis of the qualities thereof) and of single-grade tobacco leaves by thermal analysis, but also greatly reduces the workload and the number of tests, provides concrete formula design objectives, rich data support, and digital technical methodology for the development of cigarette products, and realizes the automatic search and objective evaluation of tobacco formula design schemes. It can avoid subjective factors and different representations that occur in traditional reliance on expert experience and sensory evaluation.

### EXAMPLES

The present invention is further explained by embodiments below, but is not limited by the present embodiments. Experimental methods not specified in the embodiments are generally available commercially in accordance with conventional conditions, conditions described in the manual, or general equipment, materials, reagents, etc. used in accordance with conditions suggested by the manufacturer, unless otherwise specified. The following embodiments and the raw materials in the applicable ratios are commercially available.

Example: Analysis method of composition and proportion of tobacco leaf groups in a well-known domestic brand cigarette product sample (to be analyzed), the steps are as follows:

(1) A product sample of a well-known domestic brand of cigarettes (i.e., the cigarette to be analyzed) and 50 single-grade tobacco samples of different origin, different parts and different grades of 5 grams each (e.g., of the cigarette to be analyzed and of each of the single-grade tobacco leaves) were selected. The cigarette to be analyzed and the single-grade tobacco leaf samples were screened with a 100-mesh screen and treated for 48 hours in a constant temperature and humidity environment of (22±1)° C. and (60±2) % relative humidity.

(2) Before the sample thermogravimetric analysis, the thermogravimetric analyzer was set and kept at 900° C. for 10 min to clear the impurities in the furnace body, and the empty crucible was used as a reference. A (5.00±0.05) mg sample was weighed and placed in a platinum thermogravimetric crucible, and the heating procedure was as follows: an initial temperature of 50° C., a heating rate of 10° C./min to a final temperature of 900° C., and holding at a constant temperature of 900° C. for 5 min. The protection gas and reaction gas were nitrogen, and the flow rate was 20 mL/min. Taking temperature (° C.) as one axis (e.g., the X-axis) and mass change (%) as an orthogonal axis (e.g., the Y-axis), a graph of the percentage change in mass of the sample plotted as a function of temperature (e.g., the exported data) is TG result data.

(3) The first derivative of the weight data (i.e., the TG result data) with respect to time was taken to obtain a differential weight loss curve data (e.g., a DTG matrix), a DTG matrix Y for the cigarette to be analyzed, and DTG matrices for the single-grade tobacco leaves X=[X₁ X₂ . . . . X₅₀].

TABLE 1
Cigarette DTG matrix Y

| Temperature ° C. | 30 | 31 | . . . | 45 | 46 | . . . | 900 |
|---|---|---|---|---|---|---|---|
| Cigarette to | −2.62E−05 | −2.62E−05 | . . . | −2.81E−05 | −3.62E−05 | . . . | −5.43E−05 |
| be analyzed | | | | | | | |

TABLE 2
Single-grade tobacco DTG matrix X

| Temperature ° C. | 30 | 31 | . . . | 45 | 46 | . . . | 900 |
|---|---|---|---|---|---|---|---|
| Tobacco leaf 1 (X₁) | −3.86E−05 | −3.86E−05 | . . . | −4.07E−05 | −4.94E−05 | . . . | −4.51E−05 |
| Tobacco leaf 2 (X₂) | −2.53E−05 | −2.53E−05 | . . . | −2.73E−05 | −3.54E−05 | . . . | −5.27E−05 |
| . . . | . . . | . . . | . . . | . . . | . . . | . . . | . . . |
| Tobacco leaf 50 (X₅₀) | −2.77E−05 | −2.77E−05 | . . . | −2.96E−05 | −3.78E−05 | . . . | −4.72E−05 |

(4) The formula ratio real number coding matrix R=[r₁ r₂ . . . r₅₀] is set, wherein r₁, r₂ . . . r₅₀ represents the proportion or ratio of the 50 single-grade tobacco leaves in the formulation, as shown in Table 3:

TABLE 3
Formula proportional real number coding matrix R

| Single grade tobacco | Formula ratio rᵢ |
|---|---|
| Tobacco leaf 1 | r₁ |
| Tobacco leaf 2 | r₂ |
| . . . | . . . |
| Tobacco leaf 50 | r₅₀ |

The proportion or ratio of single-grade tobacco leaves rᵢ in the above table is initialized randomly to a real value between 0 and 1, and rᵢ is subsequently normalized to ensure that the sum of the proportion or ratio values of each tobacco leaf e.g., in the formula ratio real number coding matrix R) is 1. The formula is as follows:

$${r}_{i} = {r}_{i} / {\sum }_{i = 1}^{n} {r}_{i} ,$$

where n is 50.

TABLE 4
Random initialization results of formula
proportional real number coding matrix R

| Single grade tobacco | Formula ratio rᵢ |
|---|---|
| Tobacco leaf 1 | 0.03 |
| Tobacco leaf 2 | 0.04 |
| . . . | . . . |
| Tobacco leaf 50 | 0.06 |
| Total | 1.00 |

According to the above method, 200 real number coding matrices R₁, R₂, . . . R₂₀₀ are initialized at the same time to establish a formula library (e.g., a search space) for analytic searching, as shown in the following table:

TABLE 5
Formula proportional real number coding initialization

| Coding | R₁ | R₂ | R₃ | R₄ | . . . | R₂₀₀ |
|---|---|---|---|---|---|---|
| r₁ | 0.05 | 0.14 | 0.02 | 0.00 | . . . | 0.12 |
| r₂ | 0.02 | 0.02 | 0.01 | 0.16 | . . . | 0.14 |
| r₃ | 0.04 | 0.03 | 0.02 | 0.22 | . . . | 0.02 |
| r₄ | 0.03 | 0.05 | 0.08 | 0.06 | . . . | 0.03 |
| . . . | . . . | . . . | . . . | . . . | . . . | . . . |
| r₅₀ | 0.26 | 0.04 | 0.08 | 0.08 | . . . | 0.09 |
| Total | 1.00 | 1.00 | 1.00 | 1.00 | 1.00 | 1.00 |

The cigarette DTG matrix Z (e.g., an atlas or library of tobacco formula DTG matrices with randomly initiated values) is calculated by combining single-grade tobacco leaf DTG matrices (e.g., in or from the matrix X) according to the proportions/ratios in the formulas in the real number coding matrix R (e.g., Zᵢ=X′×Rᵢ):

TABLE 6
Cigarette fusion map matrix Z after combination

| Temperature ° C. | 30 | 31 | . . . | 45 | 46 | . . . | 900 |
|---|---|---|---|---|---|---|---|
| Z₁ | −3.23E−05 | −3.23E−05 | . . . | −3.43E−05 | −4.24E−05 | . . . | −5.99E−05 |
| Z₂ | −3.35E−05 | −3.35E−05 | . . . | −3.54E−05 | −4.32E−05 | . . . | −5.86E−05 |
| . . . | . . . | . . . | . . . | . . . | . . . | . . . | . . . |
| Z₂₀₀ | −6.27E−05 | −6.27E−05 | . . . | −6.51E−05 | −7.48E−05 | . . . | −5.47E−05 |

A DTG difference correlation model, e={square root over (((Z−Y))Σ⁻¹(Z−Y))}, is used to calculate the difference value e between Z and Y, so as to evaluate the conformity of the composition analysis of the tobacco leaf groups (e.g., to determine a difference between the 10 tobacco sample fusion map matrix Y and each of the tobacco fusion maps in the matrix Z), as shown in Table 7 below:

TABLE 7
Analytical coincidence (difference between
Z and Y) of coiled tobacco composition

| Candidate | R₁ | R₂ | R₃ | R₄ | . . . | R₂₀₀ |
|---|---|---|---|---|---|---|
| Difference e | 3.6703 | 2.5197 | 6.2708 | 2.8521 | . . . | 1.5228 |

The difference values e are converted to a probability value P(e) using the formula

$$P ( e ) = \frac{{e}_{max} - e}{{e}_{max} - {e}_{min}} / sum ( \frac{{e}_{max} - e}{{e}_{max} - {e}_{min}} ) ,$$

as shown in Table 8 below:

TABLE 8
Convert the difference value e to a probability value

| Candidate | R₁ | R₂ | R₃ | R₄ | . . . | R₂₀₀ | Total |
|---|---|---|---|---|---|---|---|
| Probability | 2.38% | 4.36% | 4.79% | 3.79% | . . . | 6.08% | 100% |
| value P(e) | | | | | | | |

The first 100 candidates for the formula ratio were selected randomly and/or according to the probability values (e.g., the tobacco fusion maps in the matrix Z having the highest probability value), and the formula ratios of the candidates were reorganized linearly in pairs (or analyzed pairwise by linear regression) according to the equation r⁽¹⁾=r₁+a*(r₁-r₂), where a is a scale factor, generated by random numbers with a uniform distribution following [−d, 1+d], and d is 0.25, a limit so that the reorganization (or regression) range to within a manageable range or distribution (e.g., so that it is not too large.

The reorganized real number coding matrix R₁⁽¹⁾, R₂⁽¹⁾, . . . R₂₀₀⁽¹⁾ is obtained as shown in Table 9 below:

TABLE 9
Real number code of formula ratio after the first reorganization

| Code | R₁⁽¹⁾ | R₂⁽¹⁾ | R₃⁽¹⁾ | R₄⁽¹⁾ | . . . | R₂₀₀⁽¹⁾ |
|---|---|---|---|---|---|---|
| r₁ | 0.00 | 0.14 | 0.40 | 0.07 | . . . | 0.19 |
| r₂ | 0.20 | 0.03 | 0.01 | 0.05 | . . . | 0.06 |
| r₃ | 0.25 | 0.25 | 0.08 | 0.02 | . . . | 0.07 |
| r₄ | 0.03 | 0.39 | 0.27 | 0.13 | . . . | 0.05 |
| . . . | . . . | . . . | . . . | . . . | . . . | . . . |
| r₅₀ | 0.07 | 0.03 | 0.11 | 0.01 | . . . | 0.07 |
| Total | 1.00 | 1.00 | 1.00 | 1.00 | 1.00 | 1.00 |

According to the real number coding of the reorganized formula proportions or ratios, a reorganized (or regressed) cigarette DTG matrix Z⁽¹⁾ was calculated. The DTG difference association model was invoked to calculate the difference value e⁽¹⁾ between Z and Y, and the difference value e was iteratively calculated (e.g., to generate e⁽²⁾, e⁽³⁾, e⁽⁴⁾, e⁽⁵⁾, . . . ) until e<0.0001 (in this example, until e=0.000095).

According to the probability value P(e) from large to small, the top 5 formula proportional candidates are output, as shown in Table 10 below:

TABLE 10
Formula analysis results and P(e) values (the top 5 candidates
with the highest probability values are selected)

| Code | R₇₅⁽¹⁾ | R₂₄⁽¹⁾ | R₁₃⁽¹⁾ | R₁₅⁽¹⁾ | R₁₈₀⁽¹⁾ |
|---|---|---|---|---|---|
| r₁ | 0.00 | 0.00 | 0.00 | 0.00 | 0.00 |
| r₂ | 0.00 | 0.03 | 0.00 | 0.08 | 0.05 |
| r₃ | 0.10 | 0.25 | 0.20 | 0.15 | 0.16 |
| r₄ | 0.15 | 0.39 | 0.18 | 0.10 | 0.27 |
| . . . | . . . | . . . | . . . | . . . | . . . |
| r₅₀ | 0.07 | 0.08 | 0.15 | 0.11 | 0.07 |
| P(e) | 70.30% | 13.91% | 4.46% | 2.24% | 1.15% |

According to the first five formula ratio candidates in the above table, tobacco leaves with a formula ratio of 0 are filtered out to obtain the complete composition and ratio of the tobacco leaf formula, as shown in Tables 11-15 below:

TABLE 11
Leaf formula corresponding to R₇₅

| Single grade tobacco | Formula ratio |
|---|---|
| Tobacco Leaf 3 | 0.10 |
| Tobacco Leaf 4 | 0.15 |
| Tobacco Leaf 7 | 0.05 |
| Tobacco Leaf 14 | 0.05 |
| Tobacco Leaf 18 | 0.10 |
| Tobacco Leaf 20 | 0.05 |
| Tobacco Leaf 25 | 0.10 |
| Tobacco Leaf 26 | 0.05 |
| Tobacco Leaf 27 | 0.05 |
| Tobacco Leaf 32 | 0.10 |
| Tobacco Leaf 35 | 0.05 |
| Tobacco Leaf 42 | 0.04 |
| Tobacco Leaf 46 | 0.04 |
| Tobacco Leaf 50 | 0.07 |

TABLE 12
Leaf formulations corresponding to R₂₄

| Single grade tobacco | Formula ratio |
|---|---|
| Tobacco Leaf 2 | 0.05 |
| Tobacco Leaf 3 | 0.12 |
| Tobacco Leaf 4 | 0.10 |
| Tobacco Leaf 14 | 0.05 |
| Tobacco Leaf 15 | 0.08 |
| Tobacco Leaf 20 | 0.05 |
| Tobacco Leaf 25 | 0.10 |
| Tobacco Leaf 26 | 0.05 |
| Tobacco Leaf 27 | 0.05 |
| Tobacco Leaf 32 | 0.12 |
| Tobacco Leaf 35 | 0.05 |
| Tobacco Leaf 42 | 0.05 |
| Tobacco Leaf 46 | 0.05 |
| Tobacco Leaf 50 | 0.08 |

TABLE 13
Leaf formulations corresponding to R₁₃

| Single grade tobacco | Formula ratio |
|---|---|
| Tobacco Leaf 3 | 0.20 |
| Tobacco Leaf 4 | 0.18 |
| Tobacco Leaf 8 | 0.08 |
| Tobacco Leaf 13 | 0.02 |
| Tobacco Leaf 17 | 0.06 |
| Tobacco Leaf 19 | 0.10 |
| Tobacco Leaf 25 | 0.02 |
| Tobacco Leaf 28 | 0.05 |
| Tobacco Leaf 30 | 0.07 |
| Tobacco Leaf 35 | 0.06 |
| Tobacco Leaf 45 | 0.03 |
| Tobacco Leaf 46 | 0.05 |
| Tobacco Leaf 50 | 0.08 |

TABLE 14
Leaf formulations corresponding to R₁₅

| Single grade tobacco | Formula ratio |
|---|---|
| Tobacco Leaf 2 | 0.08 |
| Tobacco Leaf 3 | 0.15 |
| Tobacco Leaf 4 | 0.10 |
| Tobacco Leaf 8 | 0.05 |
| Tobacco Leaf 13 | 0.06 |
| Tobacco Leaf 17 | 0.06 |
| Tobacco Leaf 19 | 0.02 |
| Tobacco Leaf 25 | 0.02 |
| Tobacco Leaf 28 | 0.05 |
| Tobacco Leaf 30 | 0.08 |
| Tobacco Leaf 35 | 0.12 |
| Tobacco Leaf 45 | 0.05 |
| Tobacco Leaf 46 | 0.05 |
| Tobacco Leaf 50 | 0.11 |

TABLE 15
Leaf formulations corresponding to R₁₈₀

| Single grade tobacco | Formula ratio |
|---|---|
| Tobacco Leaf 2 | 0.05 |
| Tobacco Leaf 3 | 0.16 |
| Tobacco Leaf 4 | 0.27 |
| Tobacco Leaf 5 | 0.03 |
| Tobacco Leaf 12 | 0.01 |
| Tobacco Leaf 14 | 0.05 |
| Tobacco Leaf 15 | 0.02 |
| Tobacco Leaf 20 | 0.02 |
| Tobacco Leaf 25 | 0.05 |
| Tobacco Leaf 26 | 0.04 |
| Tobacco Leaf 27 | 0.04 |
| Tobacco Leaf 32 | 0.06 |
| Tobacco Leaf 35 | 0.03 |
| Tobacco Leaf 42 | 0.03 |
| Tobacco Leaf 44 | 0.02 |
| Tobacco Leaf 45 | 0.02 |
| Tobacco Leaf 46 | 0.03 |
| Tobacco Leaf 50 | 0.07 |

Verification experiment: According to the five formulations shown in Tables 11-15, the corresponding single-grade tobacco leaf samples were mixed into cigarettes, and 9 sensory evaluation experts evaluated and scored the sensory quality differences between the mixed tobacco samples and the cigarette sample to be analyzed according to the gradient or scores shown in Table 16 below. The average value was taken as the actual smoking evaluation value of the quality difference(s), and the quality difference(s) were rounded and converted to the corresponding qualitative evaluation shown in Table 16.

TABLE 16
Sensory quality difference score gradient setting

| Quality | | | | | | |
|---|---|---|---|---|---|---|
| deviation | None | slight | lesser | Intermediate | big | Large |
| Score | 0 | 1 | 2 | 3 | 4 | 5 |

The smoking evaluation results are shown in Table 17 below:

TABLE 17
Verification results of sensory evaluation

| Candidate | | | Quality |
|---|---|---|---|
| formulation | P(e) | Average | difference |
| R₇₅ | 70.30% | 0.00 | None |
| R₂₄ | 13.91% | 0.66 | Slight |
| R₁₃ | 4.46% | 0.78 | Slight |
| R₁₅ | 2.24% | 1.11 | Slight |
| R₁₈₀ | 1.15% | 1.22 | Slight |

As can be seen from Table 17, the consistency between formula ratio candidate R₇₅ and the cigarette to be analyzed was 70.30%, and there was no difference in sensory evaluation results. The coincidence between candidates R₂₄, R₁₃, R₁₅, R₁₈₀ and the cigarette to be analyzed was 13.91%, 4.46%, 2.24% and 1.15%, respectively, and the results of the sensory evaluation were slightly different.

The above embodiments disclose only several embodiments of the invention, and their descriptions are more specific and detailed, but they cannot be construed as limitations on the scope of the invention. It should be noted that for ordinary technicians in the field, without deviating from the concepts of the invention, a number of derivations and improvements can be made that are within the scope of protection of the invention. Therefore, the scope of protection of the invention patent shall be subject to the attached claims.

What is claimed is:

1. A method for analyzing a composition of tobacco leaves, comprising the following steps: (1) preparing cigarette samples to be analyzed and single-grade tobacco samples; (2) collecting a thermal analysis spectrum of the cigarette samples to be analyzed and the single-grade tobacco samples; and (3) analyzing the thermal analysis spectrum to obtain the tobacco leaf composition and proportion of the cigarette samples to be analyzed, wherein step (3), analyzing the thermal analysis spectrum to obtain the tobacco composition and proportion of the cigarette samples to be analyzed, comprises the sub-steps:
Sub-step (A): calculating a first derivative of the TG result data against time to obtain a differential weight loss DTG curve, a DTG matrix Y of the cigarette samples to be analyzed, and a DTG matrix of the single-grade tobacco samples X=[X₁ X₂ . . . Xₙ], wherein n is the number of single-grade tobacco samples;
Sub-step (B): coding formula proportion: coding a real number R=[r₁ r₂ . . . rₙ] for a formula proportion of each single grade tobacco sample, where n is the number of single-grade tobacco samples;
Sub-step (C), randomly initializing a coding matrix R: initializing a value of r to a real value between 0 and 1, where a sum of the values of each coding matrix should be 1; establishing a search space according to a range of more than 10 times a number of tobacco leaves composed of the formula, and randomly initializing the coding matrix, that is: R₁, R₂, . . . ;
Sub-step (D), calculating a DTG matrix Z of single-grade tobacco leaves combined according to a formula ratio R;
Sub-step (E): calculating a difference value e between Z and Y using a DTG difference correlation model;
Sub-step (F), converting the difference value e to a probability value P(e);
Sub-step (G), according to the probability value, screening a number of formula proportions to participate in a next iteration, randomly selecting two schemes for linear reorganization: r⁽¹⁾=r₁+a*(r₁−r₂), and obtaining reorganized real number coding matrices R₁⁽¹⁾, R₂⁽¹⁾, . . . , wherein a is a scale factor generated by random numbers that obey a [−d, 1+d] uniform distribution, and d is a value that limits a scope of reorganization;
Sub-step (H), repeating sub-steps (C)-(F) for iterative searching, and iteratively calculating e⁽²⁾, e⁽³⁾, e⁽⁴⁾, e⁽⁵⁾ . . . until e is less than a certain value; and
Sub-step (I), ordering the probability value P(e) from largest to smallest, taking a number of formula proportions, and obtaining the tobacco composition and proportion of the cigarette samples to be analyzed.

2. The analytical method of the cigarette leaf group composition of claim 1, wherein step (1) includes a single cigarette sample to be analyzed and at least 50 single-grade tobacco leaf samples; each sample is placed in a constant temperature and humidity environment of (22±1)° C. and (60±2) % relative humidity for at least 48 hours.

3. The analytical method of the cigarette leaf group composition of claim 1, wherein step (2), collecting the thermal analysis spectrum, comprises the following sub-steps: placing samples respectively in thermogravimetric crucibles (TG) and heating according to a procedure including an initial temperature at 50° C., a heating rate of 10° C./min, a final temperature of 900° C., and a constant temperature at 900° C. for 5 min using a protection gas and reaction gas comprising nitrogen at a flow rate of 20 mL/min; taking temperature (° C.) as an X-axis and mass change (%) as a Y-axis, deriving TG data, wherein exported data is TG result data.

4. The analytical method of the cigarette leaf group composition of claim 1, wherein an initialization formula for sub-step (C) is as follows:
$${r}_{i} = {r}_{i} / {\sum }_{i = 1}^{n} {r}_{i} .$$

5. The analytical method of the cigarette leaf group composition of claim 1, wherein in sub-step (D), the cigarette DTG matrix Z after combining single-grade tobacco leaves according to the formula ratio R is calculated using a calculation formula as follows: Zᵢ=X′×Rᵢ, wherein Rᵢ is an i-th random coding matrix, X is the single-grade tobacco sample DTG matrix, and Zᵢ is the DTG matrix of single-grade tobacco leaves combined according to the formula ratio in the coding matrix Rᵢ.

6. The analytical method of the cigarette leaf group composition of claim 1, wherein a formula for calculating the difference value e in sub-step (E) is as follows: e=\sqrt{(Z−Y)Σ⁻¹(Z−Y)}, wherein Y is the DTG matrix of the cigarette samples to be analyzed, Z is the DTG matrix of single-grade tobacco leaves combined according to the formula ratio, and > is a covariance matrix between Y and Z.

7. The analytical method of the cigarette leaf group composition of claim 1, wherein sub-step (F) uses a calculation formula to convert the difference value e to the probability value P(e) between 0 and 1 as follows:
$$P ( e ) = \frac{{e}_{max} - e}{{e}_{max} - {e}_{min}} / sum ( \frac{{e}_{max} - e}{{e}_{max} - {e}_{min}} ) .$$

8. The analytical method of the cigarette leaf group composition of claim 1, wherein d in sub-step (G) has a value of 0.2-0.3.

9. The analytical method of the cigarette leaf group composition of claim 1, wherein in sub-step (H), e is iteratively calculated until it is <0.0001.
